(19) United States
(12) Patent Application Publication
(10) Pub. No.: US 2010/0016219 A1
(43) Pub. Date: Jan. 21, 2010

(54) Ophthalmic compositions containing solubilized cyclosporin

(76) Inventor: Jerry Zhang, Grayslake, IL (US)

Correspondence Address: JERRY ZHANG, 1061 CHADWICK DR., GRAYSLAKE, IL, 60030, US

(21) Appl. No.: 12/218,787

(22) Filed: Jul. 19, 2008

(51) Int. Cl. A61K 38/13; A61P 27/02

(52) U.S. Cl. 514/9

(57) ABSTRACT

An ophthalmic composition containing solubilized cyclosporin is able to be obtained in the presence of at least one 1,2-alkanediol having 5 to 7 carbon atoms and at least one polysorbate surface active agent in an ophthalmically acceptable medium. The composition is stable without crystallization and/or precipitation of cyclosporin. The composition is suitable for treatment of ophthalmic disorders including keratoconjectivis Sicca and ocular rosacea.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention relates to topical compositions for treatment of ophthalmic disorders. In particular, the invention relates to ophthalmic compositions containing cyclosporin solubilized in an ophthalmically acceptable medium.

### BACKGROUND OF THE INVENTION

Cyclosporins are a group of nonpolar cyclic oligopeptides with known immunosuppressant activity, widely used in post-allogenic organ transplant. In addition, U.S. Pat. No. 4,839,342 discloses that cyclosporin (sometimes referred to in the literature as “cyclosporine” or “ciclosporin”) has been found to be effective in treating keratoconjunctivitis sicca (KCS or dry eye disease).

Cyclosporin comprises a group of cyclic oliopeptides and the major component thereof is cyclosporin A which has been identified along with several other minor metabolites, cyclosporin B through I. In addition, a number of synthetic analogs have been prepared. In general, commercially available cyclosporins may contain a mixture of several individual cyclosporins which all share a cyclic peptide structure consisting of eleven amino acids with a total molecular weight of about 1,200, but with different substitutes or configurations of some of the amino acids.

As reported, the solubility of cyclosporin in water is between 20 .mu.g/ml to 30 .mu.g/ml for cyclosporin A. Hence, the pharmaceutical formulations containing cyclosporin have been prepared as oily solutions dispersed in a vehicle.

U.S. Pat. No. 5,051,402 discloses a method of solubilizing cyclosporin in aqueous solution through forming inclusion complex with alpha.-cyclodextrin.

A topical emulsion of cyclosporin for treating KCS has been marketed under the trade name Restasis (Allergan, Inc., Irvine, Calif.). In this emulsion as disclosed in U.S. Pat. No. 5,474,979, cyclosporin is in an admixture with a higher fatty acid glyceride, such as castor oil, and a surface active agent, such as polysorbate 80, and an emulsion stabilizer, such as a cross-linked polyacrylate.

However, application of an emulsion containing oily droplets may result in eye irritation or a clouding of visual field. Furthermore, active ingredient is generally more bioavailable in solubilized form than in insoluble, suspended, or inclusion complex form.

For ophthalmic application, aqueous compositions containing solubilized active ingredients are generally preferred.

Therefore, there is a need for topical treatment of ophthalmic disorders by administering a composition containing solubilized cyclosporin without disadvantages of the prior art.

There is also a need for an enhanced ophthalmic composition containing solubilized cyclosporin.

### SUMMARY OF THE INVENTION

It has been surprisingly discovered that the ophthalmic compositions containing solubilized cyclosporin can be obtained in the presence of at least one polysorbate surface active agent and at least one 1,2-alkanediol having 5 to 7 carbon atoms in an ophthalmically acceptable medium.

Accordingly, it is an object of the invention to formulate stable aqueous ophthalmic compositions containing solubilized cyclosporin.

Another object of the invention is to formulate enhanced aqueous compositions for treating ophthalmic disorders such as KCS and ocular rosacea.

Still other objects and advantages of the invention will, in part, be obvious and will, in part, be apparent from the following detailed description of the preferred embodiments.

### DETAILED DESCRIPTION OF THE PREFERRED EMBODIMENTS

In the described embodiments, disclosed are stable ophthalmic compositions containing solubilized cyclosporin.

The term ‘stable’, as used herein, means physical, rather than chemical stability. In accordance with the present invention, the cyclosporin compositions are stable, that is substantially no crystallization and/or precipitation in the compositions, when stored at a refrigerated temperature for at least 14 days. The refrigerated temperature, in accordance with the present invention, is a temperature in the range of just above the freezing temperature of the aqueous composition, which is about 0.degree. C., to about 10.degree. C.

The term ‘dissolved’, ‘dissolving’, ‘solubilized’ or ‘solubilizing’, when used in accordance with the present invention, means that an ingredient is substantially solubilized in the aqueous composition, and that the ingredient will not exist to any appreciable degree in the particulate, crystalline or droplet form in the composition.

As used herein, the term ‘about’ will be understood by persons of ordinary skill in the art and will vary to some extent on the context in which is used. If there are uses of the term which are not clear to persons of ordinary skill in the art given the context in which is used, ‘about’ will mean up to plus or minus 10% of the particular term.

The term ‘ophthalmically acceptable’, as used herein, refers to those compounds, materials, compositions, and/or dosage forms which are, within the scope of sound medical judgment, suitable for use in contact with a mammalian eye without excessive toxicity, irritation, allergic response, or other problem or complication, commensurate with a reasonable benefit/risk ratio.

The term ‘safe and effective’, as used herein, means a concentration of an active ingredient or an amount of a composition, that is sufficient enough to significantly and positively modify the condition to be treated but low enough to avoid serious side effects, within the scope of sound medical advice.

The term ‘cyclosporin’ or ‘cyclosporins’, as used herein, is meant to include not only cyclosporin A, but also those analogs of cyclosporin, namely cyclosporin B through I, which are substantially solubilized in the compositions of the present invention and which have therapeutic activity when topically applied. The term ‘cyclosporin’ or ‘cyclosporins’, as used herein, is also meant to include cyclosporin either prepared from synthetic method or isolated from natural source, either in admixture or in pure or substantially pure form. All physical forms of cyclosporin, crystalline, semi-crystalline, and amorphous, are contemplated and within the scope of the present invention, either in admixture or in pure or substantially pure form.

All percentages referred to in this specification are percentages by weight of the total composition unless otherwise indicated.

The ophthalmic compositions of the present invention may contain solubilized cyclosporin within the concentration range of about 0.01% to about 5%, preferably about 0.02% to about 2%, more preferably about 0.05% to 2%.

1,2-Alkanediols are alcohols having two hydroxyl groups. The alkyl chain in the 1,2-alkanediols in accordance with the present invention is a saturated, straight alkyl group having from 5 to 7 carbon atoms. These 1,2-alkanediols include 1,2-pentanediol, 1,2-hexanediol, 1,2-heptanediol, and any suitable combinations thereof. Furthermore, the stereoisomers of 1,2-alkanediols are also contemplated and within the scope of the present invention. The definition of 1,2-alkanediols in accordance with the present invention embraces all possible stereoisomers and their mixtures. It particularly embraces the racemic forms and the isolated optical isomers having the specified activity.

These 1,2-alkanediols are commercially available, for example, from Aldrich Chemical Company (Milwaukee, Wis.). They may also be chemically synthesized, for example, by catalytic hydroxylation of the corresponding terminal alkenes (also known as 1-alkenes) using a combination of hydrogen peroxide and osmium tetroxide.

1,2-Hexanediol is preferred.

Concentration of the 1,2-alkanediol in the composition of the present invention may be in the range of about 0.1% to about 10%, preferably about 0.2% to about 5%, more preferably about 0.5% to about 2%.

Polysorbate (also commercially known as Tween) family of surface active agents, as used herein, is nonionic emulsifiers derived from polyoxyethylene sorbitan esterified with fatty acids. The polysorbate family of surface active agents in accordance with the present invention includes polysorbate 20, polysorbate 40, polysorbate 60, polysorbate 80, polysorbate 85, and any suitable combination thereof.

Polysorbate 80 is preferred.

In one embodiment, concentration of the polysorbate in the composition of the present invention may be in the range of about 0.01% to about 5%.

In another embodiment, concentration of the polysorbate in the composition of the present invention may be in the range of about 0.02% to about 5%.

In yet another embodiment, concentration of the polysorbate in the composition of the present invention may be in the range of about 0.05% to about 2%.

In still another embodiment, concentration of the polysorbate in the composition of the present invention may be in the range of about 0.1% to about 2%.

In order to provide that the aqueous ophthalmic formulation achieves minimal eye irritation, it is important that for a certain concentration of cyclosporin in the ophthalmic formulation, lower concentration of a polysorbate is preferred while maintaining solubilization of cyclosporin.

The formulations of the present invention include an ophthalmically acceptable medium, preferably an ophthalmically acceptable liquid aqueous medium. This medium often acts as a vehicle or medium, e.g., as a solvent, for the other components in the formulation. One particularly useful ophthalmically acceptable medium is water. Preferably, the medium, and in fact the entire formulation, is sterile.

In order to provide that the aqueous ophthalmic formulation does not irritate one's eye, it is important that the ophthalmic formulation have a pH value in the range of about 6.5 to about 8, preferably about 6.8 to about 7.5, and still more preferably so that the pH of the ophthalmic formulation substantially corresponds to the pH value of the fluids in the eye, in particular, the human eye.

To stabilize or maintain the ophthalmic formulation at the desired pH, an effective minor amount of at least one buffer component is incorporated into the ophthalmic formulation. The effective minor amount of buffer component employed to buffer or maintain the formulation at the desired pH can vary widely and depends to a large degree on the particular buffer component employed, as well as the chemical composition of the ophthalmic formulation.

Any suitable buffer component can be employed which is compatible with the other ingredients of the ophthalmic formulation, and which does not have deleterious or toxic properties which could harm the eye. Examples of suitable ophthalmically acceptable buffer components include acetate buffers, citrate buffers, phosphate buffers, borate buffers, and combinations thereof. Specific buffer components useful in the present invention include boric acid, sodium borate, sodium citrate, and sodium phosphates, including mono, di-, and tri-basic phosphates, such as sodium phosphate monobasic and sodium phosphate dibasic, and combinations thereof. Conventional organic buffers, such as TRIS (tromethamine) buffer, Good's buffer (e.g., HEPES buffer) and the like, may also be employed.

It should be noted that any other suitable ophthalmically acceptable buffer components can be employed to maintain the pH of the ophthalmic formulation so that the ophthalmic formulation is provided with an acceptable pH, and the before-mentioned buffer components are merely examples of such buffer components.

When it is determined that the buffered ophthalmic formulation does not have the desired pH value, the pH of the aqueous buffered ophthalmic formulation can be adjusted by the addition of an effective amount of either a base or an acid, as the case may be. Any suitable base or acid can be employed to adjust the pH of the aqueous buffered ophthalmic formulation which does not provide the ophthalmic formulation with toxic or deleterious properties which could harm either ophthalmic devices or the eye. An example of a base which can be used to adjust the pH of the aqueous buffered ophthalmic formulation is sodium hydroxide; and an example of an acid which can be used to adjust the pH of the aqueous buffered ophthalmic formulation is hydrochloric acid.

Further, in order to provide that the present ophthalmic formulations do not irritate the eye, e.g., the eye of the wearer of the contact lens treated using such formulations, it is important that the ophthalmic formulations have an osmolality (a measure of tonicity) of at least about 200 mOsmol/kg, preferably in the range of about 200 to about 350 or about 400 mOsmol/kg. In an especially useful embodiment, the osmolality or tonicity of the formulation substantially corresponds to the tonicity of the fluids of the eye, in particular the human eye.

Any suitable ophthalmically acceptable tonicity component or components may be employed, provided that such component or components are compatible with the other ingredients of the ophthalmic formulation and do not have deleterious or toxic properties which could harm the eye. Examples of useful tonicity components include sodium chloride, potassium chloride, mannitol, dextrose, glycerin, propylene glycol, and combinations thereof. In one embodiment, the tonicity component is selected from inorganic salts and suitable combinations thereof.

The amount of ophthalmically acceptable tonicity component utilized can vary widely. In one embodiment, the tonicity component is preferably present in the ophthalmic formulation in an amount in the range of about 0.5% to about 0.9%.

In another embodiment, the ophthalmic formulation contains glycerin in an amount in the range of about 0.5% to about 5%, preferably about 1% to about 5%, more preferably about 2% to about 3%.

Typical of ophthalmically acceptable inorganic salt tonicity components are alkali metal chlorides and alkaline earth metal chlorides, such as sodium chloride, potassium chloride, calcium chloride, and magnesium chloride.

The ophthalmic formulation in accordance with the present invention may contain polyethylene glycol (PEG). Examples of the suitable PEG include PEG 200, PEG 300, PEG 400, PEG 600, PEG 1000, PEG 1500, and combinations thereof. The amount of PEG included in the present compositions containing such a component preferably is in the range of about 0.01% to about 10%, preferably about 0.05% to about 5%.

Ophthalmic products are typically packaged in multidose form. Preservatives are thus required to prevent microbial contamination during use. Suitable preservatives include: benzalkonium chloride, Polyquan preservative (Alcon), perborate (e.g., sodium perborate), Purite preservative (stabilized chlorine dioxide) (Allergan), thimerosal, chlorobutanol, methyl paraben, propyl paraben, phenylethyl alcohol, edetate disodium, sorbic acid, Onamer M, or other agents known to those skilled in the art.

The amount of preservative component included in the present compositions containing such a component varies over a relatively wide range depending, for example, on the specific preservative component employed. The amount of such component preferably is in the range of about 0.000001% to about 0.05% or more of the present composition.

Ophthalmic products may also be packaged in single-use dosage form.

One or more additional components can be included in the present formulations based on the particular application for which the formulations are made. Thus, the present formulations can be made up as disinfecting compositions, cleaning compositions, wetting compositions, conditioning compositions, soaking compositions and the like. Also, the present formulations can be made up to be useful in performing two or more contact lens caring operations. For example, a disinfecting/cleaning formulation, or a cleaning/conditioning composition or even an all purpose lens care formulation can be made up and such multi-functional formulations are included within the scope of the present invention.

The ophthalmic formulation in accordance with the present invention may contain compounds having at least one amide bond. Examples of these compounds include urea, N-2-hydroxyethyl urea, niacinamide, isonicotinamide, caffeine, N-picolylnicotinamide, N-allylnicotinamide, and suitable combinations thereof. The amount of these compounds included in the ophthalmic formulations containing such a component preferably is in the range of about 0.01% to about 10%, preferably about 0.05% to about 5%.

The additional component or components included in the present formulation are chosen to impart or provide at least one beneficial or desired property to the formulations. Such additional components may be selected from components which are conventionally used in one or more ophthalmic compositions. Examples of such additional components include cleaning agents, wetting agents, nutrient agents, chelating agents, viscosity builders, antioxidants, and the like. These additional components are each included in the present formulations in an amount effective to impart or provide the beneficial or desired property to the compositions.

Examples of useful wetting agents include polyvinyl alcohol, polyoxamers, polyvinyl pyrrollidone, carboxyl methylcellulose, hydroxypropyl methyl cellulose, and suitable combinations thereof.

Examples of useful chelating agents include EDTA (ethylenediaminetetraacetic acid), EGTA [ethylenebis(oxyethylenenitrilo)tetraacetic acid], and ophthalmically acceptable salts thereof, alkali metal hexametaphosphate, citric acid, sodium citrate, and suitable combinations thereof.

Examples of useful viscosity builders include hydroxyethyl cellulose, hydroxypropyl cellulose, hydroxypropyl methyl cellulose, polyvinyl pyrrolidone, polyvinyl alcohol, and suitable combinations thereof.

Examples of useful antioxidants include sodium metabisulfite, sodium thiosulfate, N-acetylcysteine, butylated hydroxyanisole, butylated hydroxytoluene, and suitable combinations thereof.

The aqueous composition of the present invention may be used for topical treatment of ophthalmic disorders that are responsive to therapy with cyclosporin. In accordance with the method of treatment of the present invention, a stable aqueous composition containing solubilized cyclosporin is topically administered in a safe and effective amount to ocular surfaces of human eye in need of such therapy.

The therapeutic method of the present invention may be used to treat any ophthalmic disorder that is responsive, or potentially responsive, to cyclosporin therapy. Examples of the disorders that are suitable to be treated include inflammation in the human eye, and certain immune eye diseases that may be treated topically.

Preferably, the ophthalmic disorders to be treated are KCS and ocular rosacea.

The following examples are included for purposes of illustrating the technology covered by this disclosure. They are not intended to limit the scope of the claimed invention in any manner. One skilled in the art will understand that there are alternatives to these specific embodiments that are not completely described by these examples.

### EXAMPLE 1

The compositions in accordance with the present invention are subject to a physical stability test. The prepared compositions are packaged in glass vials and sealed. The sealed vials are kept at room temperature for 24 hours and monitored for crystal formation and/or precipitation. After 24 hours, the vials are placed in a refrigerator maintained at a temperature of about 4.degree. C. for at least 14 days. After 14 days, the vials are removed from the refrigerator and allowed to warm up to room temperature. Evidence of crystal formation and/or precipitation in the compositions is examined. The test results are recorded: “clear” means that no evidence of crystal formation and/or precipitation is evident; “crystals formed” or ‘precipitates formed’ means that crystals and/or precipitates are formed in the compositions.

### EXAMPLE 2

This example is for comparison.

| | Amount |
|---|---|
| Component | (weight percentage) |
| Cyclosporin A | 0.05% |
| Glycerin | 2.95% |
| Polysorbate 80 | 0.7% |
| Water | q.s. 100% |

The formulation was prepared by combining the ingredients in water. Cyclosporin was not soluble in the formulation.

### EXAMPLE 3

This example is for comparison.

| | Amount |
|---|---|
| Component | (weight percentage) |
| Cyclosporin A | 0.05% |
| Hexylene glycol | 0.95% |
| Glycerin | 2.0% |
| Polysorbate 80 | 0.7% |
| Water | q.s. 100% |

The formulation was prepared by combining the ingredients in water. Cyclosporin was not soluble in the formulation.

### EXAMPLE 4

This example is for comparison.

| | Amount |
|---|---|
| Component | (weight Percentage) |
| Cyclosporin A | 0.05% |
| PEG 400 | 0.95% |
| Glycerin | 2.0% |
| Polysorbate 80 | 0.7% |
| Water | q.s. 100% |

The formulation was prepared by combining the ingredients in water. Cyclosporin was not soluble in the formulation.

### EXAMPLE 5

This example is to prepare an ophthalmic formulation in accordance with the present invention.

| | Amount |
|---|---|
| Component | (weight percentage) |
| Cyclosporin A | 0.05% |
| 1,2-Hexanediol | 0.95% |
| Glycerin | 2.0% |
| Polysorbate 80 | 0.7% |
| Water | q.s. 100% |

The formulation was prepared by combining the ingredients in water. Cyclosporin was solubilized in the formulation. The pH value of the solution was adjusted to about 7.4. It was then packaged and sealed in a glass vial. The stability test described in Example 1 was performed and the test results were recorded.

The formulation remained clear and no evidence of crystal formation and/or precipitation was evident. Therefore, the formulation was found to be stable.

The results in Examples 2, 3, 4, and 5 clearly demonstrate that 1,2-alkanediol plays an unique role in solubilization of cyclosporin when compared to other organic compounds having at least two hydroxyl groups. The discovery in the present invention is unexpected.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

1-20. (canceled)

21. A homogenous liquid ophthalmic composition suitable for topical administration to an eye, comprising by weight of total composition:
cyclosporin in an amount within the range of 0.01% to 5%;
at least one 1,2-alkanediol having from 5 to 7 carbon atoms in an amount within the range of 0.1% to 10%;
at least one polysorbate in an amount within the range of 0.01% to 5%;
an ophthalmically acceptable aqueous medium.

22. The composition of claim 21 wherein the 1,2-alkanediol comprises 1,2-hexanediol.

23. The composition of claim 21 wherein the polysorbate comprises polysorbate 80.

24. The composition of claim 21 wherein the cyclosporin comprises cyclosporin A.

25. The composition of claim 21 wherein the cyclosporin comprises cyclosporin A, the polysorbate comprises polysorbate 80, and the 1,2-alkanediol comprises 1,2-hexanediol.

26. The composition of claim 21 wherein the 1,2-alkanediol is selected from the group consisting of 1,2-pentanediol, 1,2-hexanediol, 1,2-heptanediol, and combinations thereof.

27. The composition of claim 21 wherein the polysorbate is selected from the group consisting of polysorbate 20, polysorbate 40, polysorbate 60, polysorbate 80, polysorbate 85, and combinations thereof.

28. A homogeneous liquid ophthalmic composition suitable for administration to an eye, comprising by weight of total composition:
cyclosporin A in an amount within the range of 0.01% to 5%;
1,2-hexanediol in an amount within the range of 0.1% to 10%;
polysorbate 80 in an amount within the range of 0.01% to 5%
an ophthalmically acceptable aqueous medium;
wherein pH of the composition is adjusted to a value within the range of 6.8 to 7.5.

29. A homogenous liquid aqueous composition for topical treatment of an ophthalmic disorder, comprising by weight of total composition:
cyclosporin in an amount within the range of about 0.01% to 5%;
at least one 1,2-alkanediol having from 5 to 7 carbon atoms in an amount within the range of 0.1% to 10%;
at least one polysorbate in an amount within the range of 0.01% to 5%;
an opthalmically acceptable aqueous medium.

30. The composition of claim 29 wherein the ophthalmic disorder is keratoconjectivis Sicca.

31. The composition of claim 29 wherein the 1,2-alkanediol comprises 1,2-hexanediol.

32. The composition of claim 29 wherein the polysorbate comprises polysorbate 80.

33. The composition of claim 29 wherein the cyclosporin comprises cyclosporin A.

34. The composition of claim 29 wherein the cyclosporin comprises cyclosporin A, the 1,2-alkanediol comprises 1,2-hexanediol, and the polysorbate comprises polysorbate 80.

35. The composition of claim 29 wherein the polysorbate is selected from the group consisting of polysorbate 20, polysorbate 40, polysorbate 60, polysorbate 80, polysorbate 85, and combinations thereof.

36. The composition of claim 29 wherein the 1,2-alkanediol is selected from the group consisting of 1,2-pentanediol, 1,2-hexanediol, 1,2-heptanediol, and combinations thereof. [chemical structure]
